(19) United States
(12) Patent Application Publication
(10) Pub. No.: US 2013/0171104 A1
(43) Pub. Date: Jul. 4, 2013

(54) SYNERGISTIC ACTIVITY OF MODULATORS OF THE NO METABOLISM AND OF NADPH OXIDASE IN THE SENSITIZATION OF TUMOR CELLS

(71) Applicant: Georg Bauer, Freiburg (DE)

(72) Inventor: Georg Bauer, Freiburg (DE)

(73) Assignee: Universitaetsklinikum Freiburg, Freiburg (DE)

(21) Appl. No.: 13/818,089

(22) PCT Filed: Aug. 10, 2011
(86) PCT No.: PCT/EP11/63742

§ 371 Date: Feb. 20, 2013

(30) Foreign Application Priority Data
Aug. 20, 2010 (EP) 10173500.9

(51) Int. Cl. A61K 38/21; A61K 31/05; A61K 31/352; A61K 31/105; A61K 38/18; A61K 31/337

(52) U.S. Cl. CPC A61K 38/217; A61K 38/18; A61K 31/337; A61K 31/352; A61K 31/105; A61K 31/05
USPC 424/85.5; 514/7.6; 514/449; 514/456; 514/707; 514/733

(57) ABSTRACT

What is disclosed is pharmaceutical compositions which contain a pharmaceutically active amount of at least one active substance which increases the available NO concentration in the cell, together with at least one active substance which stimulates the NADPH oxidase.

The subject-matter of the present application consists of agents that can be used to fight tumours. These are in this case active substances, which intervene specifically in the metabolism of the tumour cells and lead to apoptosis of the tumour cells.

Kim et al. (2009), Anticancer Res., S. 3733-3740, disclose how both Capsaicin and resveratrol can contribute to cell death in colon carcinoma cells. This publication does not give any indication of the involvement of superoxide anions, the line taken by the explanation being based on the induction of NO synthase, which is demonstrated for both substances and the combination of these and which clearly differs from the teaching of NOX stimulation by resveratrol disclosed here. Neither does this work give any indication of a synergistic effect of modulators of the NO metabolism and NOX stimulators, in the manner forming the basis for the present application. In the work by Kim et al. there is no indication of a process specifically directed against tumour cells.

US 2010/0124576 describes a combination of L-arginine and resveratrol in gels to stimulate sexual sensations. An interaction between the two substances is not backed up by data. Anti-tumour effects are not mentioned.

US 2009/163580 discloses a combination of resveratrol and quercetin in anti-aging agents. However, no data is presented to suggest a synergistic interaction between the two substances. Neither is there any reference to anti-tumour effects.

US 2008/0248129 describes various substances, to which an effectiveness against tumours is ascribed. The data do not offer any evidence of a synergistic effect of resveratrol and quercetin or other natural substances stated.

WO 2005/082407 discloses the use of quercetin and resveratrol for the treatment of oral forms of cancer. The application is limited to tumours of the oropharynx and to a purely topical application.

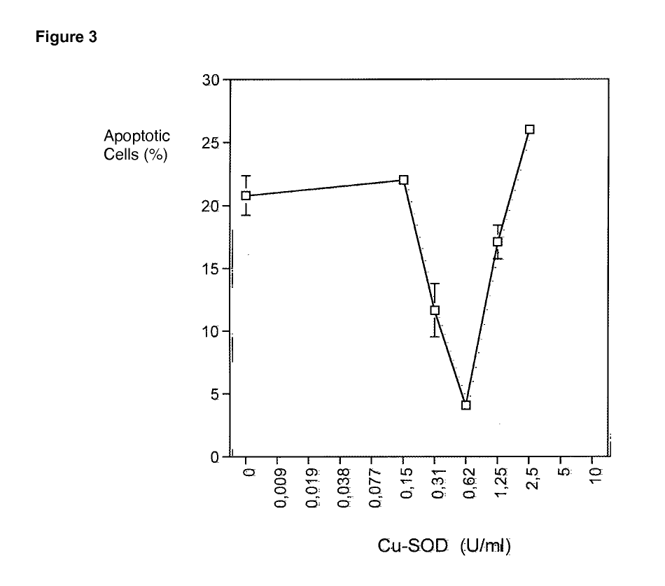

Schlachtermann et al., Translational Oncology (2008), p. 19-27 illustrate in FIG. 3 a synergistic effect on the inhibition of breast cancer cell proliferation in vitro. This synergistic effect can be demonstrated in a concentration of 0.5 μm of each individual substance in triple combination. Data on each combination of two substances are not shown, and so it is not possible from the data provided to conclude whether resveratrol plays a role in the synergistic effect of the triple combination, even if resveratrol at a higher concentration itself induces inhibition of proliferation. This work merely investigates the inhibition of the proliferation of tumour cells, without any indication of apoptosis, whereas the data according to the invention are always focussed on cell death through apoptosis. This distinction is of central importance, since while inhibiting proliferation can influence tumour growth, the destruction and elimination of a tumour, and thus the actual healing sought, requires the induction of cell death.

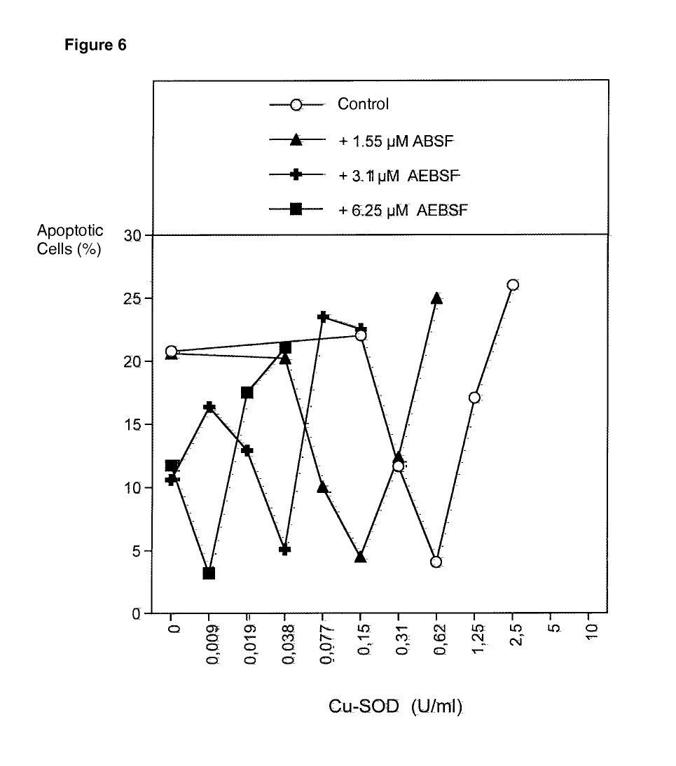

FIG. 6 shows how tumour growth can be very significantly inhibited by a combination of polyphenols as a function of their concentration. Since again no individual substances or any double combinations were tested, the result presented in FIG. 6 cannot serve as a basis for inferring a synergistic effect of combining resveratrol and quercetin, for the measured effects could also have resulted from the interaction between quercetin and catechin, without any involvement of resveratrol.

El Attar et al. (1999), Anticancer Drugs, p. 187-193 demonstrate how resveratrol and quercetin mutually strengthen their proliferation inhibiting effect on certain tumour cells. There is no indication of the induction of apoptosis under the conditions set out here and therefore the statements are of no immediate relevance, because different biological phenomena are involved, namely the inhibition of cell division as opposed to inhibition of cell death. Nor do the data provide an answer to the question of whether a synergistic effect exists in the inhibition of proliferation, if resveratrol and quercetin are applied together, since in the testing of the individual substances the concentration used in the synergy approach of 50 μm resveratrol is left out of consideration and so the effect of this concentration in administration alone cannot be inferred with certainty from the two tested concentrations (10 and 100 μM).

Jhumka et al. (2009), Int. J. of Biochem. & Cell Biology, p. 945-956, show how resveratrol in non-malignant cells (myoblasts) regulates the expression of the Na⁺/H⁺-exchanger NHE-1. In the repression caspase-3 and 6 are involved, independently of parallel apoptosis induction and hydrogen peroxide. A source of the hydrogen peroxide production is not identified. No indication is given of stimulation of NOX with the consequence of increased superoxide anion production, from which then through dismutation hydrogen peroxide could result. Unlike with the present invention in Jhumka et al. effects in normal cells are described, the relevance of which for tumour cells with their specific NOX-expression cannot be identified. Furthermore, the hydrogen peroxide molecule of such significance postulated by Jhumka et al. would have no significance in the absence of free superoxide anions in the system in question.

Wang et al. (2007), Eur. J. of Pharmacology, p. 26-35, describe effects on normal fibroblasts and not on tumour cells, wherein proliferation is dealt with rather than apoptosis.

Guha et al. (2010), The Journal of Pharmacology and Experimental Therapeutics, p. 381-394 describe the apoptosis induction in tumour cells by resveratrol and hydroxystilbene-1. Apoptosis is essentially achieved following an effect on the calcium concentration via the mitochondrial apoptosis route, without the involvement of death receptors. As a consequence of the effect of resveratrol and hydroxystilbene, rather than being the cause of this, ROS generation typical of the mitochondrial apoptosis route takes place, which necessarily results from the membrane depolarisation of the mitochondria, since the respiratory chain no longer functions under these conditions and therefore the electrons convert immediately to oxygen, and superoxide anions form, which are then converted by mitochondrial SOD into hydrogen peroxide. Neither a synergistic effect of resveratrol and other substances, nor an initial superoxide anion production, nor intracellular signalling, is triggered.

Guha et al. (2010), BJP, p. 726-734, show how an ulcer-induced effect of resveratrol is inhibited, if previously arginine is applied. In contrast to the effect according to the invention of arginine (NO synthase substrate) and resveratrol (NOX stimulator) here the apoptosis induction is a process that works in the opposite direction. The biological system investigated here does not have any identifiable direct relevance to tumour cell apoptosis.

Bechtel and Bauer (2009), Anticancer Research, p. 4559-4570, use the catalase inhibitor 3-aminotriazole, and the extra-cellular production of hydrogen peroxide by glucose oxidase or the production of superoxide anions by xanthine oxidase, in order to study how the modulation of the concentration of certain signal molecules affects the intercellular apoptosis induction of tumour cells following catalase inhibition and with which tools this can be analysed. There is no modulation of pathways which lead to a singlet oxygen mediated deactivation of the catalase. On the contrary, the catalase in the experiment is inhibited in a defined manner by 3-AT or overwhelmed by an excess of exogenously added products, but is not deactivated by a singlet oxygen-dependent process. The use of exogenously generated NO is limited to the consumption reaction of hydrogen peroxide by NO. Otherwise NO dependent signalling pathways have no part to play in this context.

WO 2008/071242 concerns active substances, which cause the available NO concentration to rise thereby leading to singlet oxygen formation. Synergy effects of the NO-increasing and NOX-activating substances are not disclosed in this application, however.

Heigold et al. (2002), Carcinogenesis, p. 929-941, describe the broad lines of NO mediated apoptosis in superoxide anion-producing transformed cells, wherein peroxynitrite formed from NO and superoxide anions represents the active apoptosis inducer. Tumour cells or their catalase, which are relevant to the present invention are not investigated in this application. None of the cell lines used in this publication are protected by catalase against peroxynitrite. From this publication, therefore, it is impossible to predict the effect of tumour cells (with protective catalase).

The present invention proceeds from the assumption that the autocrine, through reactive oxygen species (ROS) mediated apoptosis induction in transformed rat fibroplasts (208Fsrc3), as a model for cancerous cells is inhibited initially by small concentrations of Cu- or Mn-containing superoxide dismutase (Cu SOD or Mn SOD). This is based on the necessary involvement of superoxide anions in the apoptosis induction determined by the HOCl pathway. Central inhibitors such as taurine (HOCl), ABH (peroxidase) and mannitol (hydroxyl radical) substantiate the effect of the HOCl pathway. Once the maximum inhibitory effect has been achieved by both forms of the SOD then, however, concentration-dependent apoptosis induction at a higher concentration level takes place specifically for the Cu SOD. This is based on the particular electrochemical capabilities of the Cu ion in the enzyme. In the first reaction step Cu⁺⁺ SOD reacts with the one superoxide anion with the formation of oxygen and the enzyme intermediate with a monovalent copper ion:

Cu⁺⁺ SOD+O₂⁻→Cu⁺ SOD+O₂ 1)

In the second reaction step the enzyme intermediate forms hydrogen peroxide from a second superoxide anion and two protons and the starting form of the enzyme with bivalent copper is restored:

Cu⁻ SOD+O₂⁻+2H+→Cu⁺⁺ SOD+H₂O₂ 2)

At a high Cu SOD concentration not every Cu⁺ SOD intermediate finds a superoxide anion. Alternatively for this it performs with HOCl a Fenton-like reaction, in which an electron from the intermediate is transferred to HOCl and in this way the Cu⁺⁺ form of the enzyme is restored, but from HOCl chloride ions and apoptosis-triggering hydroxyl radicals the result is:

Cu⁺ SOD+HOCl→Cu⁺⁺ SOD+Cl⁻+.OH 3)

Reaction 3) is therefore responsible for the increase again in the apoptosis induction at higher Cu SOD concentrations, as the inhibitor data show. Mn SOD cannot perform this reaction since the manganese ion, unlike copper and iron ions, is incapable of the Fenton reaction.

The formation of a bell curve through the inhibition by Cu SOD is not only of interest from a radical chemistry point of view, but also provides the basis for quantifying superoxide anions. For from the fundamentals of the reaction it can be inferred that each change in the superoxide anion concentration in the system should lead to an easily detectable shift in the bell curve, wherein the apex represents a good tool for accurate measurement.

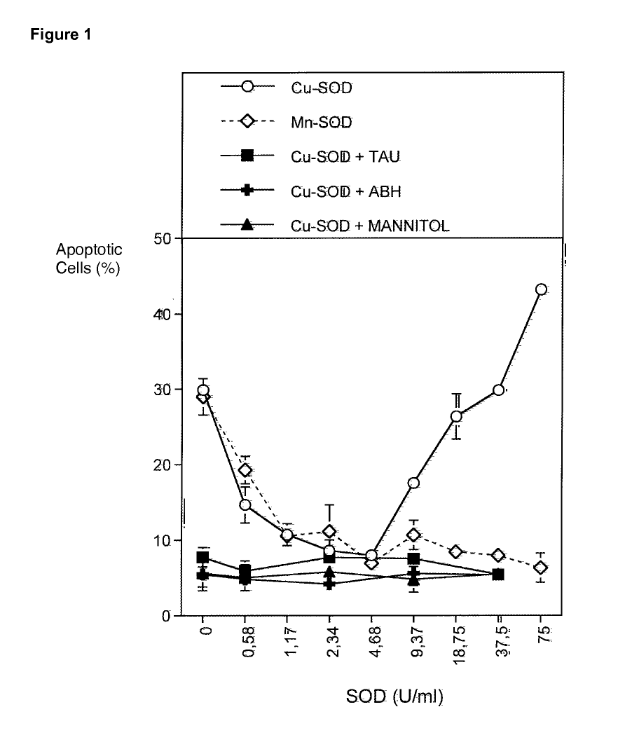

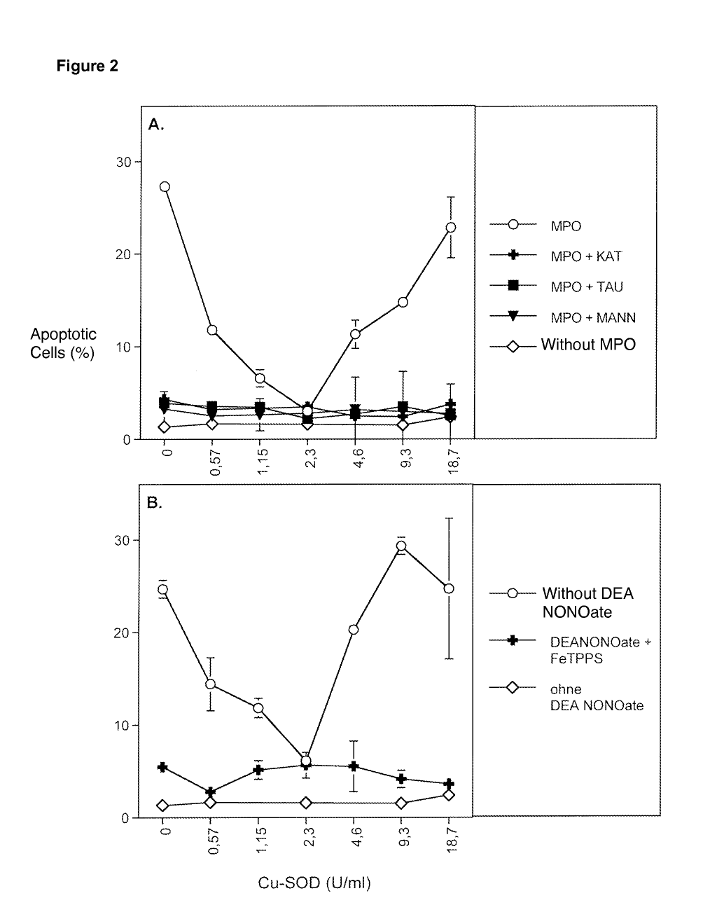

FIGS. 2A, 2B and 3 are further testament to this characteristic reaction of the Cu SOD. In FIG. 2A the HOCl pathway for 208Fsrc cells is forced by the addition of exogenous myeloperoxidase (MPO) and thus compared with the autocrine apoptosis induction without exogenous additions shown in FIG. 1 is substantially accelerated (an advantage from the testing and technical point of view). The chemical processes of the experiments shown in FIGS. 1 and 2A are identical.

In the experiment shown in Example 2, FIG. 2B in 208Fsrc3 cells, through the addition of an NO donor (DEANONOATE) the NO/peroxynitrite pathway is induced. Here again the characteristic bell curve results for Cu SOD. However, the chemistry behind this differs from the abovementioned examples cited. On the left side of the curve Cu SOD inhibits the formation of peroxynitrite, in that it takes away the superoxide anions necessary for the reaction with NO. In the right side of the bell curve the intermediate with the monovalent copper leads to the reduction of NO to the nitroxyl anion since it cannot obtain a second superoxide anion. This reacts with oxygen in the air to form peroxynitrite, which triggers apoptosis:

Cu⁺⁺ SOD+O₂⁻→Cu⁺ SOD+O₂ 1)

Cu⁺ SOD+NO→Cu⁺⁺ SOD+NO⁻ 2)

NO⁻+O₂→ONOO⁻ 3)

The example shown in FIG. 3 of the Cu SOD bell curve is based on the as yet unpublished HOCl synthesis of high concentrations of the salen-manganese complex EUK-8 (manganese N,N′-bis(salicylidiene)-ethylenediamine chloride), which is actually known as a catalase mimetic.

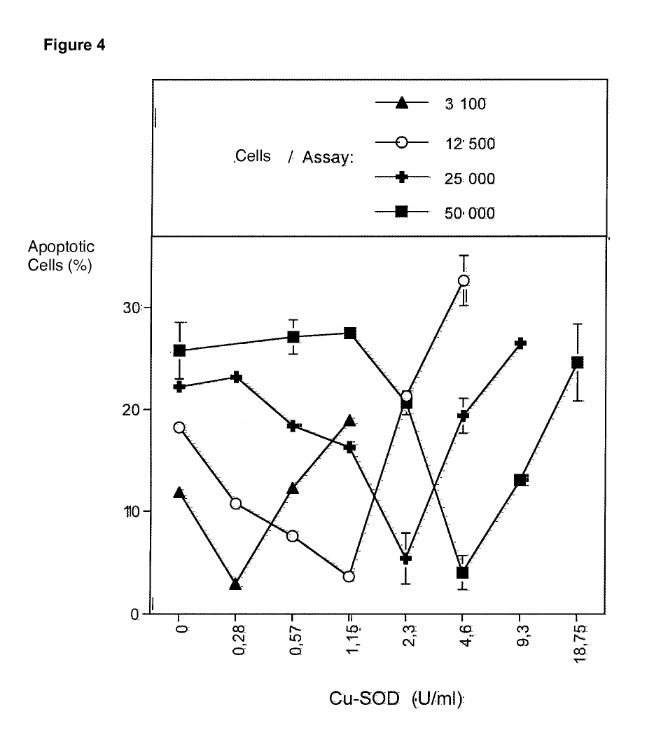

In high concentrations the substance therefore has an effect like that of an HOCl-synthesised peroxidase. The advantage of this system lies in the fact that this substance demonstrates a higher affinity for the substrate hydrogen peroxide than natural peroxidases. The reaction therefore also takes place at very low hydrogen peroxide concentrations. The measurements that are shown in FIG. 4 or 6 are possible only due to this specific apoptosis inductor.

Thus there are various apoptosis induction systems available, which can meet the various experimental requirements for the determination of the superoxide anion involvement and its associated concentration. Here the effect of the Cu SOD (but not of the Mn SOD) in the same direction in the various systems, is evidence that the explanation should be applicable to the chemical processes based on the monovalent Cu SOD.

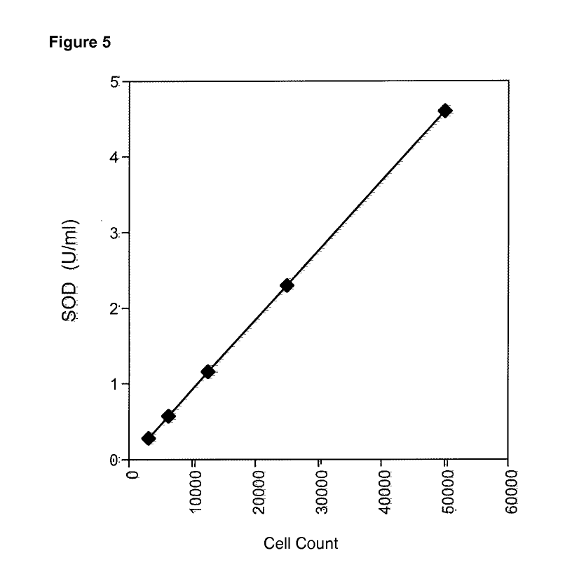

With FIG. 4 a calibration curve for measurement of the relative superoxide anion concentration by SOD can be prepared. The preparation contained various quantities of superoxide anion-producing cells, and thus varying concentrations of superoxide anions. The bell curves of the inhibition by Cu SOD were displaced as expected. The calibration curve shown in FIG. 5, determined from FIG. 4, demonstrates strict linearity.

A second confirmation of the effectiveness of this measurement system is shown in FIG. 6. Here the partial inhibition of the NADPH oxidase by AEBSF (4-(2-aminoethyl)-benzenesulfonyl fluoride) leads to clear displacements of the curves, which could also be expected due to the inhibition of the NADPH oxidase.

The application of the measurement of the superoxide anion concentration by Cu SOD is confirmed in the following examples and used to check whether certain effects or substances influence the superoxide anion production by NADPH oxidase.

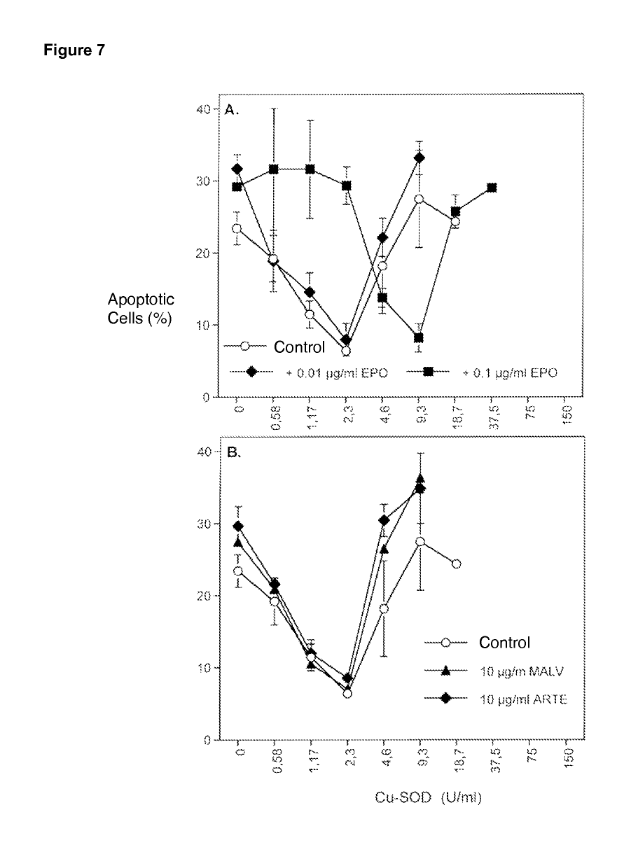

FIG. 7 shows how at the characteristic concentration of epothilone B for optimum efficiency a certain stimulation of the NADPH oxidase can be established (fourfold), while at lower concentrations this is no longer the case. (Taxol, not shown, also behaves in the same way). Substances such as malvidin chloride or artemisinin do not demonstrate any effect on the NADPH oxidase, however.

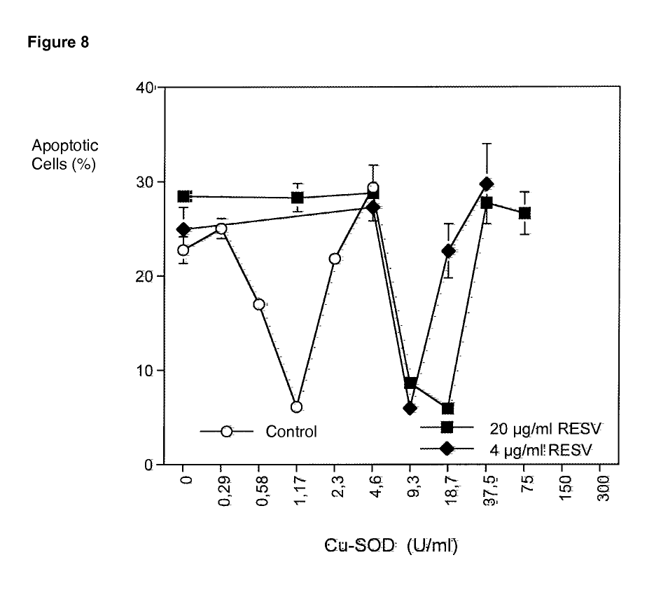

Resveratrol demonstrates very strong stimulation of the NADPH oxidase (FIG. 8).

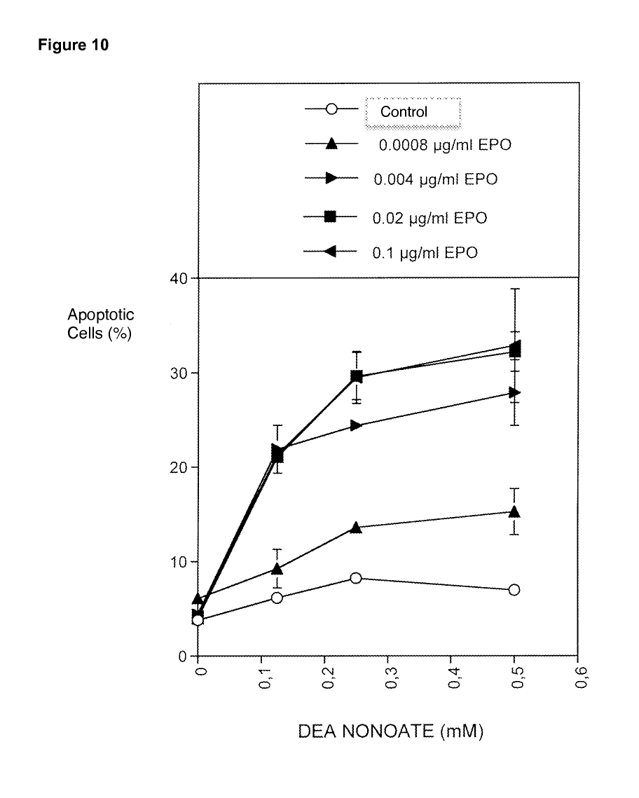

Finally, FIG. 10 demonstrates how the activation of the FAS receptor by monoclonal antibodies against the receptor (A) or by singlet oxygen (B), generated by exposure to light of the photosensitiser Photofrin, leads to a significant stimulation of the NADPH oxidase. This statement is strengthened by the fact that the inhibition of the caspase-8 immediately downstream of the FAS receptor leads to the inhibition of stimulation of the NADPH oxidase.

Inhibition of NO Dioxygenase (NOD)

This test is based on the fact that NOD also effectively converts exogenously added NO into nitrate and thus in suitable cell systems (such as for example the tumour cell line MKN-45) can prevent apoptosis induction by NO/peroxynitrite.

A precondition is a dense cellular structure of the tumour cells, the catalase of which is completely inhibited by the addition of 200 mM 3-AT. This rules out a test substance having an influence over the reaction as a whole, through modulation of the catalase activity. The hydrogen peroxide released following the catalase inhibition is fully decomposed by 20-25 μM of the catalase mimetic EUK-134 (similar to the abovementioned EUK-8, but with a lower peroxidise activity). In this way both the HOCl pathway and the consumption of NO by hydrogen peroxide are prevented. For the modulation of the available NO concentration with the known mechanisms now only the NOD remains. If this is inhibited then the addition of exogenous NO leads to an increased apoptosis induction.

FIG. 10 shows how epothilone B (EPO) over a very wide range of concentrations is highly effective in bringing about an increase in the available NO concentration, which is best explained by NOD inhibition.

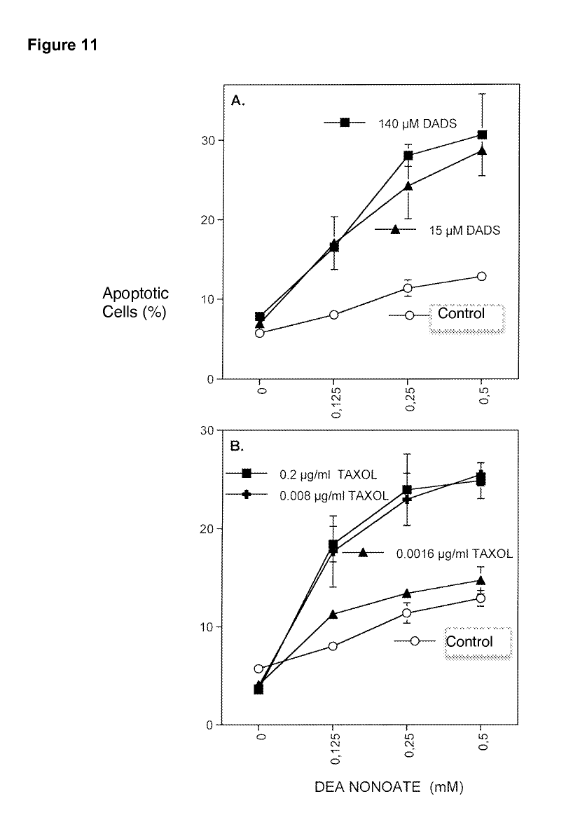

The same finding is made for diallyl disulfide (DADS) and Taxol (FIG. 11). For resveratrol there were no indications of an inhibitory capability of the NOD (data not shown). In a preferred configuration diallyl disulfide and/or Taxol are used in the combinations of active substances.

The subject matter of the present invention thus comprises pharmaceutical compositions containing a pharmaceutically active amount of at least one active substance, which increases the available NO concentration in the cell, together with an active substance that stimulates the NADPH oxidase.

In a preferred configuration the active substance, which stimulates the NADPH oxidase, is selected from among resveratrol, transforming growth factor-beta (TGF-β) and/or angiotensin II. TGF-β is one of the signalling molecules. The TGF-β polypeptides are multifunctional and can influence cell proliferation. Angiotensin II is an octapeptide and is one of the tissue hormones.

In a particularly preferred configuration the pharmaceutical composition contains as the active substance, which stimulates the NADPH oxidase, the compound resveratrol. Resveratrol is an active substance belonging to the polyphenols with anti-oxidant properties. From a chemical aspect resveratrol is a stilbenoid. Resveratrol occurs as a trans- or cis-isomer. According to the invention both isomers are used. Resveratrol is found in various plants or foodstuffs which have been obtained from such plants. Grapes, raspberries, plums and peanuts merit special mention.

According to the invention, it is preferable that the active substance that increases the available NO concentration in the cell does not have a simultaneous effect on the NADPH oxidase.

The other active substance in the pharmaceutical compositions according to the invention is an active substance which increases the NO concentration in the cell. Such an active substance can be selected from arginine and/or arginase inhibitors, in particular NOHA and/or nor-NOHA.

In a further preferred configuration the active substance, which increases the available NO concentration in the cell, is a substance, which induces the NO synthase.

A preferred active substance, which induces the NO synthase, is interferon γ.

In a further preferred configuration the active substance, which increases the available NO concentration in the cell, is a substance that has an inhibiting effect on the NO dioxygenase and is selected from among
a) flavonoids, in particular xanthohumol, isoxanthohumol, 6-prenylnaringenin, 8-prenylnaringenin, quercetin, quercitrin, isoquercetin, rutin, taxifolin, hyperosid, and/or
b) anthocyans, in particular cyanidin chloride, malvidin chloride, malvidin-3-O-galactoside, pelargonin, peonidin chloride, pelargonidin, and/or
c) fatty acids, in particular palmitic aid, stearic acid, myristic acid, and/or
d) azoles, in particular biconazole, econazole, fluconazole, itraconazole, ketoconazole, miconazole, sulconazole, and/or
e) artemisinin, chloroquin, primaquin.

The compositions according to the invention are preferably used for treating gastric cancer, prostate cancer and/or breast cancer.

The examples and Figures show synergistic effects of active substances, which increase the available NO concentration and ones which stimulate the NADPH oxidase.

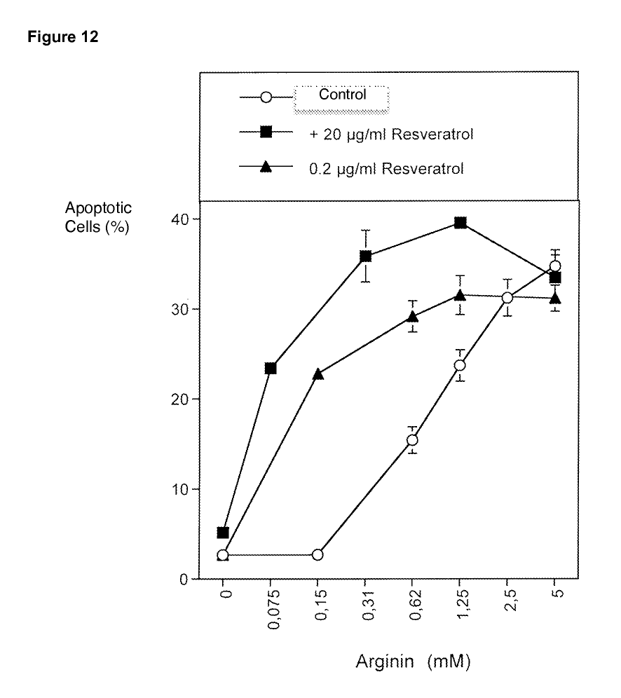

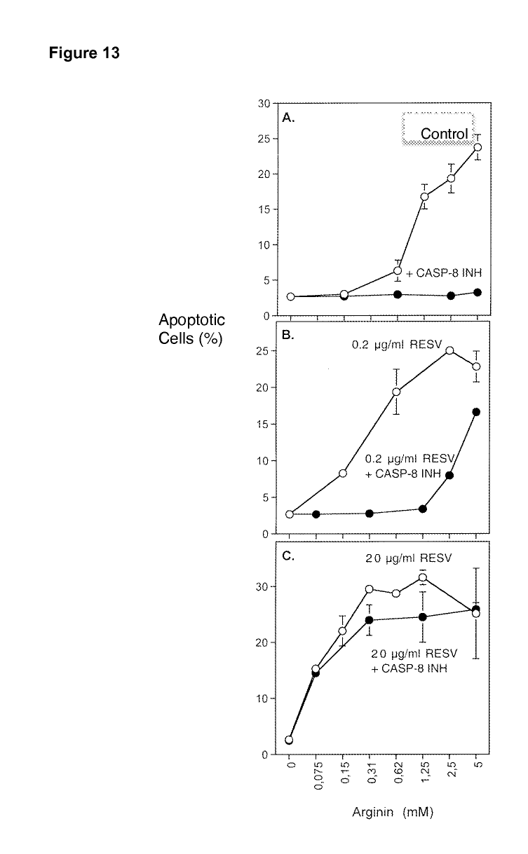

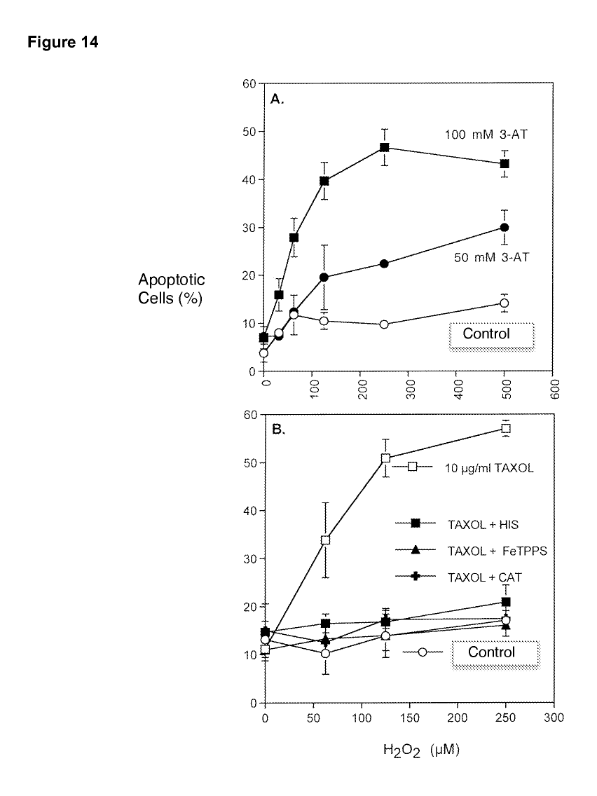

FIGS. 12 and 13 demonstrate how an increase in the arginine concentration (substrate of the NO synthase) induces apoptosis. Data demonstrating that this is based on an increase in the NO level, augmented peroxynitrite formation, singlet oxygen formation and deactivation of the catalase are not shown here. At low concentrations of arginine a highly remarkable synergistic effect with resveratrol can be identified (FIG. 12). FIGS. 13 and 14 demonstrate how the effect of arginine alone is dependent upon the amplification by the FAS system (increase in the superoxide anion production), since caspase-8 inhibitors can completely block the arginine-mediated apoptosis induction. Resveratrol can replace this FAS-dependent amplification step.

The singlet oxygen-mediated deactivation of the tumour cell catalase following the effects of Taxol is demonstrated directly in Example 14. Peroxynitrite (which can be inhibited by FeTPPS) and hydrogen peroxide (which can be inhibited by catalase) and their known reaction product singlet oxygen (inhibited by histidine) are responsible for this.

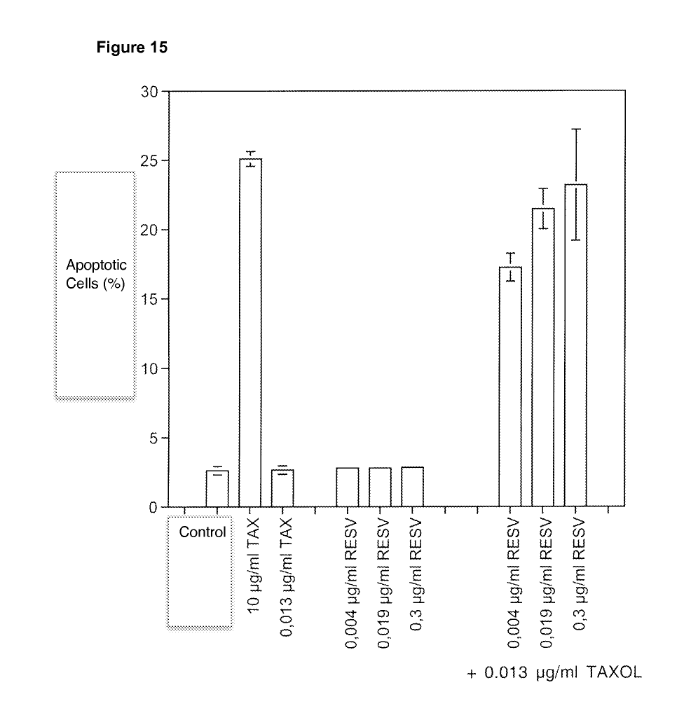

A synergistic effect with Taxol also occurs with the NADPH oxidase stimulator resveratrol (FIG. 15).

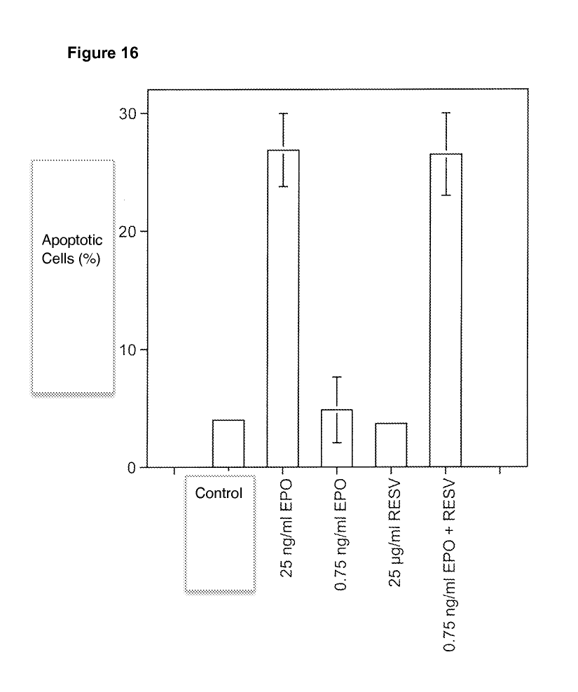

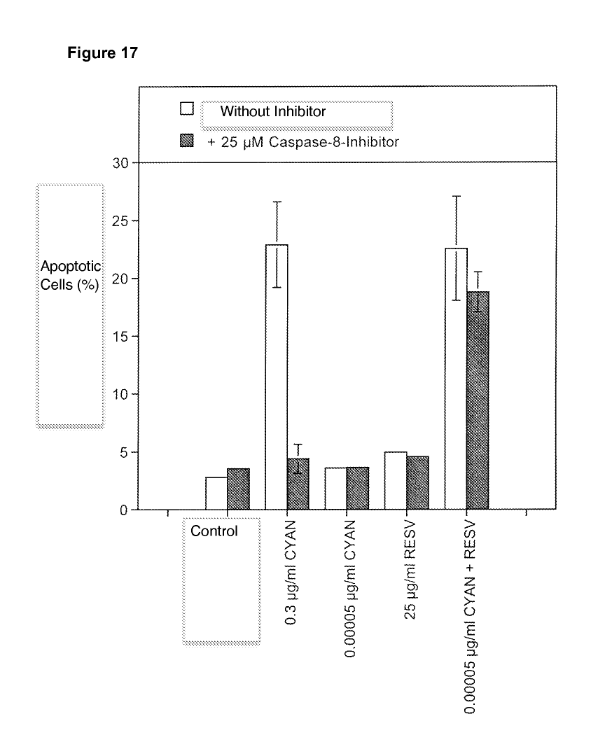

Further examples of synergistic effects are:
FIG. 16: epothilone B (NOD inhibitor) and resveratrol (NADPH oxidase stimulator);
FIG. 17: Cyanidin chloride (NOD inhibitor) and resveratrol (NADPH oxidase stimulator).

An increase in the available NO concentration through inhibition of the NOD was noted in a broad concentration range with simultaneous stimulation of the NADPH oxidase in the higher concentration range of the substances:

Taxol, epothilone B, allyl isothiocyanate

This group of substances is characterised in that in the area of the optimum effective concentration no additional stimulation by further substances is necessary. The sensitisation of the tumour cells in the optimum concentration range of this group of substances takes place without amplification steps through the FAS receptor system. In the lower concentration range the FAS system is switched to this and synergy effects with NADPH oxidase-stimulating substances (resveratrol) are observed. This has great potential for the use of synergy effects in tumour therapy.

In a preferred configuration of the invention two active substances, which are used in combination, are employed as a hybrid molecule. This means that the two molecules have a chemically covalent bond with one another, for example via a linker molecule. The linker must be selected in such a way that the biological activity of the two molecules is not adversely affected.

### EXAMPLES

### Example 1

### Effect of Superoxide Dismutase (SOD) on the Autocrine, Through Reactive Oxygen Species (ROS) Mediated Apoptosis Induction in Transformed Cells

12 500 cells of the transformed rat fibroblast line 208Fsrc3 per 100 μl complete medium were sown in 96 hole plates. Following growth 20 ng/ml TGF-beta-1 were added to all preparations. To the preparations the stated concentrations of Cu SOD (from bovine erythroctes) or Mn SOD (from E. coli) were added. Some of the preparations received 50 mM of the HOCl receptor taurine (TAU), 150 μM of the peroxidase inhibitor 4-aminobenzoylhydrazide (ABH) or 10 mM of the hydroxyl radical scavenger mannitol. After 22 hours the percentages of apoptotic cells were determined on the basis of the conventional apoptosis characteristics of nuclear condensation, nuclear fragmentation or membrane blebbing in each case in duplicate preparations.

FIG. 1 demonstrates how 208Fsrc3 cells in the presence of TGF-beta after 22 hours exhibit autocrine apoptosis. This is brought about through the HOCl signalling pathway, since it is completely inhibited by the HOCl receptor taurine, the peroxidase inhibitor ABH and the hydroxyl radical scavenger mannitol. Increasing concentrations of the Cu SOD and the Mn SOD in the concentration range below 5 U/ml, lead to a total inhibition of the apoptosis pointing to the central role of extra-cellular superoxide anions. Higher concentrations of the Cu SOD, but not of the Mn SOD, lead to a renewed increase in the apoptosis as a function of the concentration of the Cu SOD. This destructive effect of the Cu SOD is similarly dependent upon HOCl, peroxidase and hydroxyl radicals. It can be explained by the reaction of the Cu⁺ SOD intermediate. This results if the Cu⁺⁺ original form of the SOD has reacted with just one superoxide anion and been reduced by this, wherein the superoxide anion turns to molecular oxygen. In the presence of high SOD concentrations in relation to the available superoxide anion concentration the second reaction step (Cu⁺ SOD+2H⁺+O₂⁻ gives Cu⁺⁺ SOD+H₂O₂) seems no longer to take place optimally. The reaction of Cu⁺ SOD with HOCl is favoured instead, wherein in a Fenton-like reaction one electron from the Cu⁺ SOD is transferred to HOCl, wherein then apoptosis-triggering hydroxyl radicals, chloride and the native Cu⁺⁺ SOD result. The result is a bell curve of the Cu SOD effect, with a clearly defined vertex of the maximum inhibition effect on the apoptosis. The subsequent figures show how the SOD concentration, at which the vertex is achieved, is dependent upon the concentration of superoxide anions and therefore is exceptionally well-suited for a relative determination of the superoxide anion concentration.

Mn SOD does not demonstrate this feature which is characteristic of Cu SOD, once the maximum inhibition has been reached this is maintained even if the concentration increases further. This is in the nature of the Mn ion which also, as a free ion, and unlike the copper ion, is not suited to the Fenton reaction.

### Example 2

### Effect of Cu SOD on the Apoptosis Induction Mediated By the Added Myeloperoxidase (MPO) (A) or By the NO Donor DEA NONOate (B)

Instead of the autocrine apoptosis induction illustrated in Example 1, in the experiment illustrated in Example 2 the HOCl signalling pathway is accelerated by addition of exogenous MPO (FIG. 2A) or the NO/peroxynitrite pathway is induced by addition of the rapidly decomposing NO donor DEA NONOate.

12 500 transformed 208Fsrc3 cells per preparation (96 hole plate, 100 μl medium) were sown. Under A these also received 200 mU/ml MPO, and under B additionally 1.5 mM DEA NONOate. Control preparations remained free of MPO or DEA NONOate. In Part A additionally 100 U/ml catalase (KAT), 50 mM taurine (TAU), and 10 mM mannitol (MANN) were used.

In FIG. 2B the addition took place of 25 μM of the catalytically effective peroxynitrite destroyer FeTPPS. The duplicate preparations under A were assessed after 5 hours, and those under B after just 3 hours. It is clear that the addition of MPO accelerates the HOCl pathway. The inhibitors confirm the involvement of hydrogen peroxide (inhibition by catalase), HOCl (inhibition by taurine), and hydroxyl radicals (inhibition by mannitol). Again a bell curve was obtained, the right part of which is dependent upon the availability of HOCl and the effect of hydroxyl radicals.

In part B of the test through the addition of the NO donor peroxynitrite-dependent apoptosis is induced. For this NO would have to react with the superoxide anions, which are generated extracellularly from transformed cells. This reaction can of course be inhibited by SOD. The right part of the bell curve, thus the destructive effect of high concentrations of Cu SOD can be explained by the fact that the Cu⁺ intermediate form of the SOD reacts with NO to form nitroxyl anion (NO⁻). This reacts with the oxygen in the air to provide the apoptosis inductor peroxynitrite. Low concentrations of Cu SOD thus inhibit the formation of peroxynitrite from NO, because they remove the superoxide anions necessary for this from the system, while higher SOD concentrations promote the formation of peroxynitrite, because they generate nitroxyl anions, which independently of superoxide anions can form peroxynitrite directly with the oxygen in the air.

### Example 3

### Bell Curve from the Effect of Cu SOD in the EUK-8-Mediated Apoptosis Induction

The catalase mimetic EUK-8 (manganese N,N′-bis(salicylidiene)ethylenediamine chloride), a salen-manganese complex also has a peroxidase action. It has been discovered that relatively high concentrations of EUK-8 are able to synthesise HOCl. Here the affinity of the EUK-8 for hydrogen peroxide is greater than the natural peroxidase. Thus the EUK-8-mediated HOCl synthesis is suitable for apoptosis induction in superoxide anion-producing cells even with limited hydrogen peroxide availability (e.g. in the presence of tumour cell catalase).

12 500 208Fsrc3 cells in 100 μl (96 hole plate) had increasing concentrations of Cu SOD added in the presence of 120 μM EUK-8. After 5 hours the percentage of apoptotic cells was determined in duplicate preparations. Preparations without EUK-8 (but with increasing SOD concentrations) at this point in time demonstrated only a background activity of less than 5 percent apoptotic cells (data not shown in the figure).

The result shows that the addition of Cu SOD leads to a bell curve with EUK-8-mediated apoptosis as well.

### Example 4

### The Bell Curve Resulting from Cu SOD is Suitable for Relative Quantification of the Superoxide Anion Concentration

The indicated cell counts (208Fsrc3) in 100 μl medium had increasing concentrations of Cu SOD added in the presence of 120 μM EUK-8. After 1.5 hours in duplicate preparations the percentages of apoptotic cells were determined. There is a direct dependency between the SOD concentration at the vertex and the number of cells per preparation. Since this in turn determines the total concentration of available superoxide anions, there is a correlation between SOD concentration at the vertex and the superoxide anions concentration achieved. The results of the test are shown in FIG. 4.

For reasons of clarity the curve for 6 250 cells has not been shown. The vertex of this was at 0.57 U/ml SOD.

### Example 5

### Calibration SOD/Superoxide Anion Concentration

The data obtained from the experiment shown in Example 4 were recorded in such a way that the cell count (and thus the relative superoxide anion concentration) was correlated with the SOD concentration necessary for the maximum inhibition (vertex). There is a strict linear correlation. This shows that this system is suitable for the relative quantification of extracellular superoxide anions. This is shown in FIG. 5.

### Example 6

### Reduction of the Superoxide Anion Concentration Following a Gradual Inhibition of the NADPH Oxidase By AEBSF

12 500 208Fsrc3 cells (100 μl) received the stated concentrations of Cu SOD and 120 μM EUK-8. In addition the stated concentrations of the NADPH oxidase inhibitor AEBSF (4-(2-Aminoethyl)-benzenesulfonyl fluoride) were added. Control preparations remained free of AEBSF. After 5 hours the percentages of apoptotic cells were determined in duplicate preparations.

The result shown in FIG. 6 illustrates how the inhibition of the NADPH oxidase by AEBSF leads to a reduction in the superoxide anion concentration, since the bell curves of the inhibition by SOD shift to the left with their vertex as a function of the concentration. Here a doubling of the inhibitor concentration leads to a fourfold reduction in the superoxide anion concentration. Concentrations of AEBSF which were outside of the measuring range shown here could not be analysed since they led to a collapse of the reaction as a whole.

It is also important that the effect of extracellular SOD and the consequence of the AEBSF effect actually define the membrane NADPH oxidase, which generates the extracellular superoxide anions, as the target structure.

### Example 7

### Effect of Epothilone B, Malvidin Chloride and Artemisinin on the Extracellular Superoxide Anion Production of MKN-45 Cells

12 500 MKN-45 tumour cells in 100 μl medium had increasing concentrations of Cu SOD added in the presence of 150 mM 3-AT. The preparations also received, as shown, epothilone B, malvidin chloride or artemisinin. After 8 hours the percentages of apoptotic cells were determined.

FIG. 7 shows how only the highest concentration of epothilone B leads to a measurable (fourfold) increase in superoxide anion production, whereas the lower epothilone concentration, as well as malvidin chloride and artemisinin did not demonstrate any effect on the superoxide anion production.

### Example 8

### Increase in Superoxide Anion Production of MKN-45 Cells By Resveratrol

12 500 MKN-45 cells per 100 μl had the stated concentrations of Cu SOD added in the presence of 120 μM EUK-8. Control preparations remained free of resveratrol. The 4 or 20 μg/ml resveratrol were added to further preparations. The assessment was made after 4 hours.

The result (FIG. 8) shows that resveratrol in the selected concentration range brought about an 8-16-times increase in superoxide anion production.

### Example 9

### Increase in the Superoxide Anion Production of MKN-45 Cells through Activation of the FAS Receptor By Means of Antibodies or Singlet Oxygen

A: 12 500 MKN-45 cells/100 μl were treated with 10 μg/ml of an FAS receptor-activating monocolonal antibody against FAS receptors, in the presence or absence of 25 μM Caspase-8 Inhibitor. Control preparations did not receive any anti-FAS antibody and were divided into preparations with and without caspase-8 inhibitor.

B: 12 500 MKN-45 cells/100 μl had 1 μg/ml Photofrin added in the presence and absence of 25 μM caspase-8-inhibitor. The additions took place in semi-darkness. Then the preparations were exposed to the light of the neon lights of the sterile workbench. This led to the generation of singlet oxygen by the photosensitiser Photofrin. 100 mM 3-AT were then added and the stated concentrations of Cu SOD [sic]. After 5 hours the percentages of apoptotic cells were determined in the duplicate preparations.

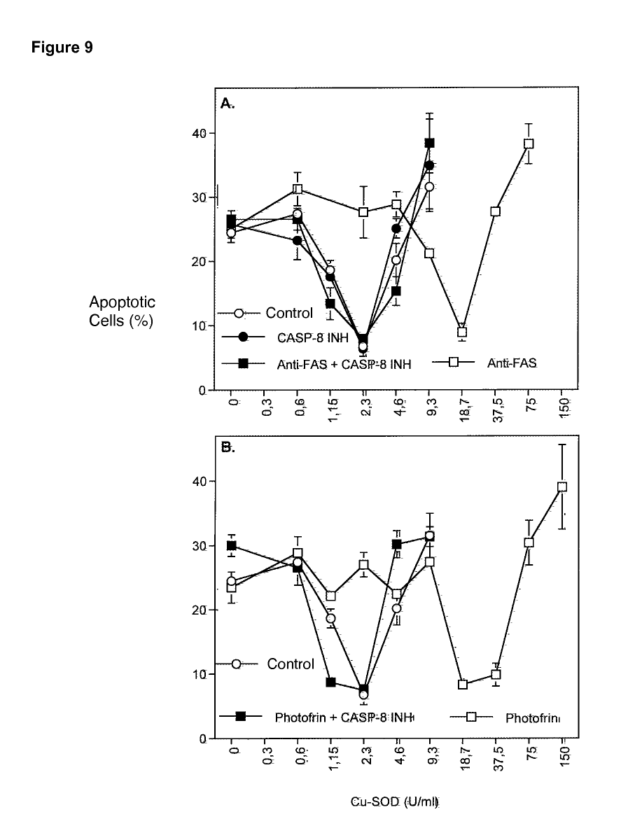

FIG. 9 shows how the activation of the FAS receptor by means of monoclonal antibodies leads to a very clear increase in the superoxide anion production. The specificity of this effect is demonstrated by the inhibition by means of caspase-8 inhibitor. Caspase-8 is activated by the FAS receptor. It is important to know that in MKN-45 cells the activation of the FAS receptor is insufficient to induce apoptosis, since the receptor density is too low.

FIG. 8 shows further how singlet oxygen demonstrates a similar effect as the monoclonal antibodies against FAS receptors. The nullification of the effect of the singlet oxygen by means of caspase-8 inhibitor demonstrates that this was mediated by the FAS receptor.

### Example 10

### Inhibition of the NO Dioxygenase (NOD) by Epothilone B

12 500 MKN-45 cells in 100 μl Medium had 200 mM 3-AT, 2.4 mM NAME, 25 μM EUK-134 and the stated concentrations of epothilone B (“EPO”) added. Control preparations did not receive any epothilone. Then the stated concentrations of the NO donor DEANONOate were added and the preparations were incubated for a further 2 hours at 37° C. before the percentages of apoptotic cells were determined.

FIG. 10 shows how all the concentrations of epothilone B used here led to an increase in the DEA-NONOATE-dependent apoptosis. This can be explained by the inhibition of the consumption of NO by the NOD. The result of this is an increased availability of NO in the system.

### Example 11

### Inhibition of the NOD By Taxol and Diallyl Disulfide (DADS)

The experiment was carried out in the same way as described in FIG. 11, but with the difference that the stated concentrations of DADS or Taxol were used and the determination of the apoptotic cells took place after 3 hours.

FIG. 11 shows how the DADS and Taxol also led to an increase in the available NO concentration. This can be explained by inhibition of the NOD.

### Example 12

### Synergistic Effect of Resveratrol and Arginine in the Sensitisation of Tumour Cells for Apoptosis Induction

12 500 MKN-45 cells in 100 μl medium were prepared with the stated concentrations of arginine in combination with 0.2 or 20 μM resveratrol. Control preparations received arginine at between 0 and 5 mM, but remained free of resveratrol. After 4.5 hours the percentages of apoptotic cells were determined (duplicate preparations).

FIG. 12 shows how the arginine (the substrate of the NO synthase) leads to a concentration-dependent apoptosis induction in the tumour cells. [Control experiments carried out in parallel (data not shown here) demonstrate how this is brought about by restoring the intercellular ROS signalling following destruction of the protective, membrane catalase of the tumour cells. In the destruction singlet oxygen generated from peroxynitrite and hydrogen peroxide plays a central and very early role].

Resveratrol, which in the concentration range selected and after 4.5 hours induces little more than background apoptosis, together with low arginine concentrations, leads to a very impressive synergistic effect. This is based on the interaction of the stimulation of the NADPH oxidase by resveratrol and the increase in the NO synthesis by arginine.

### Example 13

### Role of FAS in Apoptosis Induction By Arginine in the Absence and Presence of Resveratrol

The experiment was carried out as described in Example 12. In addition, 25 μM caspase-8 inhibitor were added or not added to the stated combinations of arginine and resveratrol. Assessment after 4.5 hours.

FIG. 13 shows how the apoptosis-triggering effect by arginine alone is strictly dependent upon the involvement of a caspase-8-mediated step. At 0.2 μg/ml resveratrol the effect of high concentrations of arginine is independent on the FAS receptor and its downstream caspase-8, while at the smaller arginine concentrations this dependency continues to exist. Finally, at 20 μg/ml resveratrol in combination with all arginine concentrations, an extensive independence from FAS and Caspase-8 is demonstrated.

### Example 14

### Direct Evidence of the Catalase Deactivation of Tumour Cells Brought About By Taxol with the Involvement of Singlet Oxygen

A: Demonstration of the protective effect of the catalase in FIG. 14A

25 000 cells of the human lymphoma line Gumbus/100 μl medium had the stated concentrations of hydrogen peroxide added without 3-AT or in the presence of 50 mM or 100 mM of the catalase inhibitor 3-AT. After 1.5 hours in duplicate preparations the apoptosis induction was determined.

B: Gumbus cells were pre-incubated without Taxol (control) or with 10 μg/ml Taxol for 30 minutes at 37° C. Parallel preparations were either free of further substances or contained 2 mM histidine (singlet oxygen-receptor), 25 μM FeTPPS (catalytically-acting peroxynitrite destroyer) or 25 U/ml catalase (CAT). Following pre-incubation the cells were separated by centrifugation, absorbed in fresh medium and the stated concentrations of hydrogen peroxide added. After 1.5 hours the assessment was performed in the duplicate preparations.

FIG. 14 shows how Gumbus have a clear protection against exogenous hydrogen peroxide, on the basis of their catalase, since this can be reversed by the catalase inhibitor 3-AT.

Pre-treatment with Taxol has the same effect as 3-AT. The inhibiting effect is also maintained once the Taxol has been washed away and is therefore best explained as an irreversible deactivation. Here singlet oxygen plays a central role. The interaction of hydrogen peroxide and peroxynitrite represents the most likely source of the singlet oxygen, as the inhibition data show.

### Example 15

### Synergistic Effect of Taxol and Resveratrol

12 500 MKN-45 cells in 100 μl had 10 μg/ml Taxol or 0.013 μg/ml Taxol in combination or not with the stated concentrations of resveratrol added. Further preparations had nothing added (control) or just the various resveratrol concentrations on their own. After 4.5 hours in duplicate preparations the percentages of apoptotic cells were determined.

FIG. 15 shows how 0.013 μg/ml Taxol or each of the stated concentrations of resveratrol in itself induced no apoptosis. 10 μg/ml Taxol demonstrated clear apoptosis induction. The combination of 0.013 μg/ml Taxol and resveratrol led to a notable synergistic effect.

### Example 16

### Synergistic Effect of Epothilone B and Resveratrol

12 500 MKN-45 cells in 100 μl medium received either no addition of substances, 25 ng/ml epothilone B, 0.75 ng/ml epothilone B, 25 μg/ml resveratrol or the combination of 0.75 ng/ml epothilone B with 25 μg/ml resveratrol. After 3 hours the percentages of apoptotic cells were determined in duplicate preparations.

FIG. 16 shows how neither the low epothilone B concentration nor resveratrol on its own was able to induce apoptosis, while in combination a synergistic effect was brought about, which achieved apoptosis induction, comparable with the high epothilone concentration.

### Example 17

### Synergistic Effect of Cyanidin Chloride and Resveratrol

12 500 MKN 45 cells in 100 μl medium received none of the stated additives. After 3 hours an assessment was made of the duplicate preparations.

FIG. 17 shows a notable synergistic effect between cyanidin chloride and resveratrol. The effect of the high cyanidin chloride concentration is dependent upon caspase-8, whereas this is barely the case for the synergistic effect.

### Example 18

### Inhibition of the Intercellular ROS Signalling By Catalase

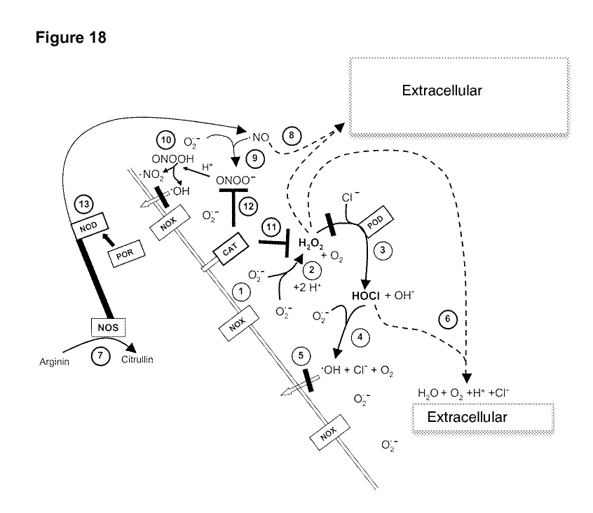

FIG. 18 shows on the left the intra-, and on the right the extracellular area of a tumour cell. The cell membrane is where the activated NADPH oxidase NOX-1 (1) is found, which generates extracellular superoxide anions. These dismute spontaneously into hydrogen peroxide and oxygen (2). In transformed cells without membrane catalase (not shown here) hydrogen peroxide is converted with a free peroxidase (POD) into HOCl (3), which reacts with superoxide anions to form apoptosis-inducing hydroxyl radicals (4, 5). With a relative excess of hydrogen peroxide there is a consumption reaction of HOCl (6). NO synthase (NOS) generates NO (7), which is either consumed by hydrogen peroxide (8) or reacts with superoxide anions to form peroxynitrite (9). Following the formation of peroxynitrous acid and its decomposition into hydroxyl radicals and NO₂ apoptosis induction (10) occurs. Tumour cells have sufficient membrane catalase, in order through the destruction of hydrogen peroxide (11) or peroxynitrite (12) to completely prevent the intercellular ROS signalling. The two lower order signalling pathways of the nitryl chloride route and the metal-catalysed Haber-Weiss reaction are not considered in the schema, but due to their dependence upon hydrogen peroxide they are likewise completely inhibited by catalase.

A key role is played by the NO dioxygenase (NOD) (13). This converts a considerable proportion of the NOS-synthesised NO into nitrate and is itself modulated by cytochrome P450 oxidoreductase (POR).

### Example 19

### Sensitisation of Tumour Cells for Intercellular ROS Signalling

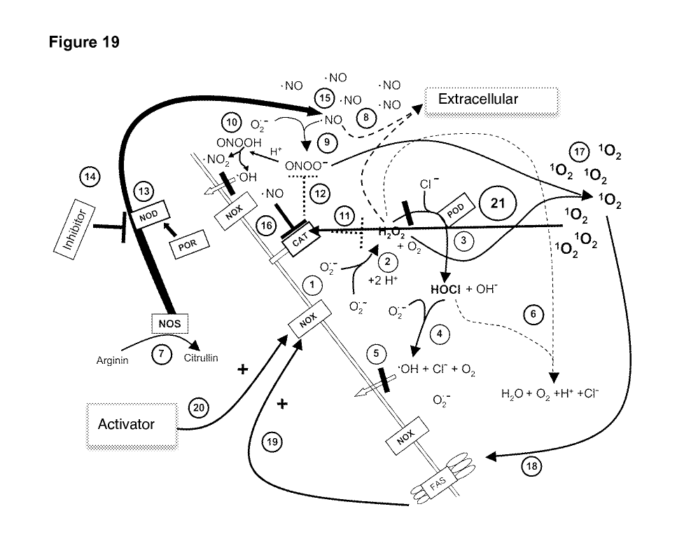

The relationship between the complex reactions is shown in FIG. 19.

The (potential) intercellular signalling pathways 1-13 correspond to those which were described in FIG. 18.

If an inhibitor of NOD occurs on a tumour cell, then there is a step increase in the available NO concentration (14, 15). The result of this is possibly a transient and partial inhibition of the catalase (16), but in any case an increase in the peroxynitrite concentration. As a consequence peroxynitrite reacts with hydrogen peroxide (17), with the formation of singlet oxygen. If this is formed in sufficient concentration, the deactivation of catalase (21) can take place immediately, as a result of which subsequently apoptosis induction through intercellular ROS signalling is enabled. If the singlet oxygen concentration is too low for the direct deactivation of catalase, then to begin with activation of the FAS receptor is carried out by singlet oxygen (18). This leads to activation of the NADPH oxidase NOX-1 (19). As a consequence the concentration of hydrogen peroxide and then that of the singlet oxygen increases and catalase is now deactivated after this amplification step. Activators of NOX-1 such as, for example, resveratrol lead to the same amplification effect as the activation of the FAS receptor (20). The same effect as through inhibition of the NOD (14) can be achieved by increasing the arginine level through addition of the amino acid or inhibition of the arginase or by induction of the expression of NOS (not shown in the schema).

The parallel increase in available NO concentration and the superoxide anion concentration could provide a new approach to the effective sensitisation and ROS-controlled self-destruction of tumour cells, in which as a result of the synergy effect the active substances can be used in a concentration range that is free from side-effects. Knowledge of the signalling pathways and the availability of corresponding test systems should also allow the synthesis of hybrid molecules which combine both the required activities in one molecule.

1. A pharmaceutical composition comprising a pharmaceutically active amount of a first active substance that increases available NO concentration in a cell in combination with a pharmaceutically active amount of a second active substance that stimulates NADPH oxidase.

2. The pharmaceutical composition according to claim 1, characterised in that the second active substance is selected from the group consisting of resveratrol, transforming growth factor β and angiotensin II.

3. The pharmaceutical composition according to claim 1, characterised in that the first active substance does not have a simultaneous effect on NADPH oxidase.

4. The pharmaceutical composition according to claim 1, characterised in that the first active substance is selected from the group consisting of arginine and arginase inhibitors.

5. The pharmaceutical composition according to claim 1, characterised in that the second active substance is resveratrol, combined with the arginase inhibitor nor-NOHA.

6. The pharmaceutical composition according to claim 2, characterised in that the second active substance is resveratrol, combined with Taxol or diallyl disulfide.

7. The pharmaceutical composition according to claim 2, characterised in that the second active substance is resveratrol, combined with cyanidin chloride.

8. The pharmaceutical composition according to claim 2, characterised in that resveratrol is combined with an azole.

9. The pharmaceutical composition according to claim 2, characterised in that the first and second active substances are in the form of a hybrid molecule.

10. The pharmaceutical composition according to claim 1, characterised in that the first active substance increases available NO concentration in the cell by inducing NO synthase.

11. The pharmaceutical composition according to claim 10, characterised in that the first active substance is interferon γ.

12. The pharmaceutical composition according to claim 1, characterised in that the first active substance has an inhibiting effect on NO dioxigenase and is selected from the group consisting of:
a) flavonoids,
b) anthocyans,
c) fatty acids,
d) azoles,
e) artemisinin, chloroquin, and primaquin.

13. A method of treating cancer in a subject in need thereof comprising the step of administering to said subject a therapeutically effective amount of the pharmaceutical composition according to claim 1.

14. The method according to claim 13, wherein said cancer is gastric cancer.

15. The pharmaceutical composition according to claim 1, characterised in that the first active substance is selected from the group consisting of NOHA and nor-NOHA.

16. The pharmaceutical composition according to claim 1, characterised in that the first active substance has an inhibiting effect on NO dioxigenase and is selected from the group consisting of:
a) flavonoids selected from among xanthohumol, isoxanthohumol, 6-prenylnaringenin, 8-prenylnaringenin, quercetin, quercitrin, isoquercetin, rutin, taxifolin, and hyperosid;
b) anthocyans selected from among cyanidin chloride, malvidin chloride, malvidin-3-O-galactoside, pelargonin, peonidin chloride, and pelargonidin;
c) fatty acids selected from among palmitic aid, stearic acid, and myristic acid;
d) azoles selected from among biconazole, econazole, fluconazole, itraconazole, ketoconazole, miconazole, and sulconazole;
e) artemisinin, chloroquin, and primaquin.
